(19) United States
(12) Patent Application Publication
(10) Pub. No.: US 2026/0035645 A1
(43) Pub. Date: Feb. 5, 2026

(54) MICROFLUIDIC TRANSWELL SYSTEMS FOR IMAGING PAIRED CELL INTERACTIONS VIA EXOSOMES

(71) Applicant: University of Florida Research Foundation, Incorporated, Gainesville, FL (US)

(72) Inventors: Niloy BARUA, Gainesville, FL (US); Michael CHUNG, Morriston, FL (US); Juan GUAN, Gainesville, FL (US); Mei HE, Gainesville, FL (US)

(21) Appl. No.: 19/101,084

(22) PCT Filed: Aug. 15, 2023
(86) PCT No.: PCT/US2023/072240

§ 371 Date: Feb. 4, 2025

Related U.S. Application Data
(60) Provisional application No. 63/398,299, filed on Aug. 16, 2022.

(51) Int. Cl. C12M 1/32; C12M 1/00; C12M 3/06; C12N 15/113; G01N 15/10; G01N 15/14; G01N 33/50; G01N 33/68

(52) U.S. Cl. CPC C12M 23/12; C12M 23/16; C12M 23/38; C12M 29/10; C12N 15/113; G01N 15/14; G01N 33/5005; G01N 33/68; C12N 2310/20; G01N 2015/1006; G01N 2333/70539; G01N 2458/00

(57) ABSTRACT

Described are microfluidic high-throughput single cell transwell systems for culturing single paired cells for real time monitoring of exosomes biogenesis, transportation and internalization between paired single cells.

### CROSS-REFERENCE TO RELATED APPLICATIONS

This application claims the benefit of U.S. Provisional Application No. 63/398,299, filed Aug. 16, 2022, which is incorporated herein by reference.

### STATEMENT REGARDING FEDERALLY SPONSORED RESEARCH OR DEVELOPMENT.

This invention was made with government support under R35 GM133794 awarded by the National Institutes of Health. The government has certain rights in the invention.

### BACKGROUND

Exosomes are nano-sized biovesicles released into surrounding body fluids upon fusion of multivesicular bodies and the plasma membrane. They were shown to carry cell-specific cargos of proteins, lipids, and genetic materials, and can be selectively taken up by neighboring or distant cells far from their release, reprogramming the recipient cells upon delivery of their bioactive compounds. Therefore, the regulated formation of exosomes, specific makeup of their cargo, cell-targeting specificity are of immense biological interest considering the potential of exosomes as non-invasive diagnostic biomarkers, as well as therapeutic nanocarriers for delivery of drug delivery vehicles.

SUMMARY

Described are microfluidic high-throughput single cell transwell systems for culturing single paired cells for real time monitoring of exosomes biogenesis, transportation and internalization between paired single cells.

The transwell system comprises two microwells, one for an exosome donor cell and one for the recipient cell, connected by a microchannel. Exosomes produced by the exosome donor cell can diffuse to the recipient cell via the microchannel. Nutrients are provided to the cells via additional microfluidic perfusion channels. The microfluidic transwell system is configured to permit fluorescence microscopic analysis of the cells.

In some embodiments, a microfluidic transwell system for use in analyzing exosome interactions comprises: a first microwell sized and adapted to culture a single cell; a second microwell sized and adapted to culture a single cell; and a microchannel connecting and in fluid communication with the first microwell and the second microwell. The first microwell and the second microwell can each be about 10 μm to about 100 μm in diameter and about 10 μm to about 120 μm in depth. In some embodiments, the first microwell and the second microwell are each about 40 μm in diameter and about 100 μm in depth. The microchannel can be about 5 μm to about 15 μm in width, about 5 μm to about 15 μm in width, and about 10 to about 60 μm in length. In some embodiments, the microchannel is about 10 μm in width and about 10 μm in width. The microfluidic transwell system is configured to permit fluorescence microscopic analysis of a cell or exosome in the first microwell, the second microwell, and/or the microchannel. In some embodiments, a microscopy coverslip forms the base or bottom of the bottom of the first microwell, the second microwell, and the microchannel to facilitate microscopic analysis.

In some embodiments, a microfluidic transwell system comprises one or more perfusion channels in fluid connection with the first microwell and/or the second microwell. The perfusion channel is adapted to allow media, nutrients or other agents to be delivered the cells in the transwell system. A perfusion channel can be about 10 μm to about 40 μm in width and about 10 μm to about 100 μm in depth. In some embodiments, a perfusion channel is about 20 μm in width. A perfusion channel can be further connected to a media inlet.

In some embodiments, two of more microfluidic transwell systems are connected by a one or more perfusion channels to form a microfluidic transwell systems array. 2, 3, 4, 5, 6, 7, 8, 9, 10, or more microfluidic transwell systems can be connected by the one or more perfusion channels. The microchannels of the two of more microfluidic transwell systems can be of the same length or different lengths.

In some embodiments, single exosome donor cell expressing a tagged endogenous protein, wherein the tagged endogenous protein is incorporated into exosomes is placed in one microwell of a transwell system and a single recipient cell is placed in the second well of a transwell system. In some embodiments, the tagged endogenous protein comprises a CRISPR-generated genomic modification of the endogenous protein. The tag can be, but is not limited to, fluorescent protein, or fragment thereof. In some embodiments, the tag is configured to be present in the interior of the exosome.

To study exosome function, an exosome donor cell (such as a dendritic cell) and a paired recipient cell (such as a T cell) are placed into a microfluidic transwell system. The donor cell is modified to express a tagged endogenous protein, wherein the tagged endogenous protein is incorporated into exosomes. The endogenous protein can be, but is not limited to, an MHC protein (e.g., a HLA protein). Labeled exosomes can be analyzed and monitored vis fluorescence microscopy. In some embodiments, the donor cell or the recipient cell comprises a patient-derived cell.

The described devices and methods can be used for high spatiotemporal resolution tracking of single exosomes at the single cell level and analyzing exosomal sorting and secretion.

### BRIEF DESCRIPTION OF THE DRAWINGS

FIG. 1. a) The schematic of endogenous protein labeling workflow. The DNA sequence coding the 11th strand of fluorescence protein is inserted at the gene of interest. As a result, a fluorescence protein is tagged to the protein of interest whenever the cell expresses this protein in the context of its native gene regulatory network. b) Representative images of endogenous protein tagging was achieved for labeling KRAS, NRAS, HRAS, BRAF, and CRAF. The scale bar is 5 μm.

FIG. 2. a) Trajectories generated from automated single particle tracking algorithm. The area shown is 50 μm×60 μm. Color scalebar codes time. b) and c) Analysis of transport dynamics: mean square displacement as a function of time, diffusivity, and self-part of van Hove function to characterize displacement distribution. d) A wavelet-based universal thresholding algorithm that can self-calibrate for distinguishing between “moving” and “stationary” phases of trajectories.

FIG. 3. Schematic illustration of endogenous tagging method over bulky antibody labeling, for retaining the natural immunogenicity of exosomes.

FIG. 4. Schematic illustration of specific MHC-I CRISPR knock in for fluorescence tagging of exosomes. Donor DNA and sgRNA sequences designed for inserting mNeonGreen tag precisely to the genomic site of MHC-I C-terminus is shown. The MHC-I homologous region is in gray; the mNeonGreen tag is in green; stop codon of the MHC-I genomic sequence is in red. DC cell line with stable endogenous MHC-I::mNeonGreen expression is generated through CRISPR/Cas9 gene editing and fluorescence activated cell sorting (FACS).

FIG. 5. Schematic illustration of microfluidic high-throughput single cell transwell system for real-time tracking of exosome immunity regulation from the single donor-recipient cell pairs.

FIG. 6. Diagram showing insert and a sequence repeat (boxed) upstream of insert. Top sequence is the HLA-A with FP11 template sequence (SEQ ID NO: 1). Bottom sequence is the FP1.RP4.upper-FP1 aligned sequence (SEQ ID NO: 2). Also shown is sgRNA 0.60 PAM TGG (SEQ ID NO: 3).

FIG. 7. Diagram showing insert. The sequence upstream of desired insert is not shown due to the use of a primer downstream of the insert. Top sequence is the HLA-A with FP11 template sequence (SEQ ID NO: 4). Bottom sequence is the FP2.RP4-RP4 aligned sequence (SEQ ID NO: 5). Also shown is sgRNA 0.60 PAM TGG (SEQ ID NO: 3).

FIG. 8A-D. (A) Fluorescence microscopic image of MHC-I::mNG U937 cells. (B) Fluorescence microscopic image of EVs secreted from MHC-I::mNG U937 cells. (C) and (D) Images illustrating real time single particle tracking of MHC-I::mNG EVs at time of 5 seconds and 30 seconds, respectively.

FIG. 9E-F. (E) Graph illustrating nanoparticle tracking analysis to characterize the EV size distribution and concentration between MHC-I::mNG U937 cells derived EVs and control U937 cells derived EVs. (F) Graph illustrating nanoview analysis of EV surface marker expression between MHC-I::mNG U937 cells derived EVs and control U937 cells derived EVs.

FIG. 10. Diagram showing workflow of the single cell droplet generation and handling for single cell dispensing into the microfluidic transwell.

FIG. 11. Diagram illustrating a single cell dispenser via microfluidic droplet system.

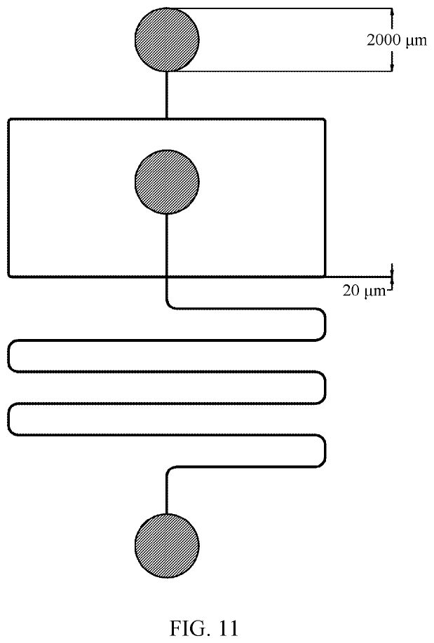

FIG. 12. Diagram illustrating transwell systems having varying microchannel lengths.

FIG. 13A. Diagram illustrating a microfluidic transwell system array having 8 transwell systems arranged in an octagon.

FIG. 13B. Diagram illustrating a multi-unit microfluidic transwell system array format.

FIG. 14. Diagram illustrating a single chip containing 40 transwell system arrays.

FIG. 15. Time-lapse live-cell fluorescence microscopic imaging showing real-time tracking of exosomes from a donor cell.

### DETAILED DESCRIPTION

### I. Definitions

Before describing the present teachings in detail, it is to be understood that the disclosure is not limited to specific compositions or process steps, as such may vary. As used in this specification and the appended claims, the singular form “a,” “an,” and “the” include plural references unless the context clearly dictates otherwise. Thus, for example, reference to “a peptide” includes a plurality of peptides and the like. The conjunction “or” is to be interpreted in the inclusive sense, i.e., as equivalent to “and/or,” unless the inclusive sense would be unreasonable in the context.

The use of “comprise,” “comprises,” “comprising,” “contain,” “contains,” “containing,” “include,” “includes,” and “including” are not intended to be limiting. It is to be understood that both the foregoing general description and detailed description are exemplary and explanatory only and are not restrictive of the teachings. To the extent that any material incorporated by reference is inconsistent with the express content of this disclosure, the express content controls.

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 0 to 20%, 0 to 10%, 0 to 5%, or up to 1% of a given value. Where particular values are described in the application and claims, unless otherwise stated the term “about” meaning within an acceptable error range for the particular value should be assumed. In the context of the lengths of nucleotide sequences, the terms “about” or “approximately” are used these lengths encompass the stated length with a variation (error range) of 0 to 10% around the value (X±10%).

All ranges are to be interpreted as encompassing the endpoints in the absence of express exclusions such as “not including the endpoints”; thus, for example, “within 10-15” includes the values 10 and 15. One skilled in the art will understand that the recited ranges include the end values, as whole numbers in between the end values, and where practical, rational numbers within the range (e.g., the range 5-10 includes 5, 6, 7, 8, 9, and 10, and where practical, values such as 6.8, 9.35, etc.). When values are expressed as approximations, by use of the antecedent “about,” it will be understood that the particular value forms a further aspect. For example, if the value “about 10” is disclosed, then “10” is also disclosed.

Membranous vesicles are released by a variety of cells into the extracellular microenvironment. Based on the mode of biogenesis, these membranous vesicles can be classified into three broad classes (i), “exosomes” (also termed extracellular vesicles (EVs)) (following the MISEV 2018 guideline, the term EVs is used to cover exosomes as a specific subpopulation of membranous vesicles that includes exosomes, and excludes ectosomes (microvesicles) and apoptotic bodies (Thery C et al. “Minimal information for studies of extracellular vesicles 2018 (MISEV2018): a position statement of the International Society for Extracellular Vesicles and update of the MISEV2014 guidelines,” J Extracell Vesicles 7(1):1535750 (2018)), (ii), ectosomes or microvesicles, and (iii) apoptotic bodies. Extracellular vesicles are cell-derived vesicles originating from endosomal compartments produced during the vesicular transport from the endoplasmic reticulum (ER) to the Golgi apparatus. Extracellular vesicles are released extracellularly after the multivesicular bodies are fused with the plasma membrane. Exosomes contain proteins from secretory cells, including those of endosome origin (e.g., ESCRTs), those involved in intracellular transport (e.g., Rab GTPase), and those of cell membrane origin (e.g., CD63 and CD81).

Extracellular vesicles are distinct from both ectosomes and apoptotic bodies in size, content, and mechanism of formation. Ectosomes are vesicles of various size (typically 0.1-1 mm in diameter) that bud directly from the plasma membrane and are shed to the extracellular space. Ectosomes have on their surface the phospholipid phosphatidylserine. Apoptotic bodies are formed during the process of apoptosis and engulfed by phagocytes. Exosomes are nanoscale membrane vesicles and contain numerous plasma membrane and cytoplasmic cell components. Dendritic cell (DC)-derived exosomes are produced from dendritic cells.

“Dendritic cells” are antigen-presenting cells having the broadest range of antigen presentation and the ability to activate naïve T cells. Their main function is to process antigen material and present it on the cell surface to T cells. DCs present antigen to both helper and cytotoxic T cells.

### II. Paired Cell Interactions

Described are devices and methods of using the devices to monitor exosome shedding, transportation, and internalization between paired single cells in real time. The devices (transwell systems and arrays or transwell systems) comprise microfluidic platforms that enable live single cell imaging and tracking of exosome biogenesis in a exosome donor cell and shedding and transport to an exosome recipient cells in one integrated workflow. The devices and methods provide for the study of cellular communications at high-precision and throughout. The devices and methods provide for improved microscopic analysis of exosome biogenesis and molecular targets with high precision and high throughput.

### III. Transwell System

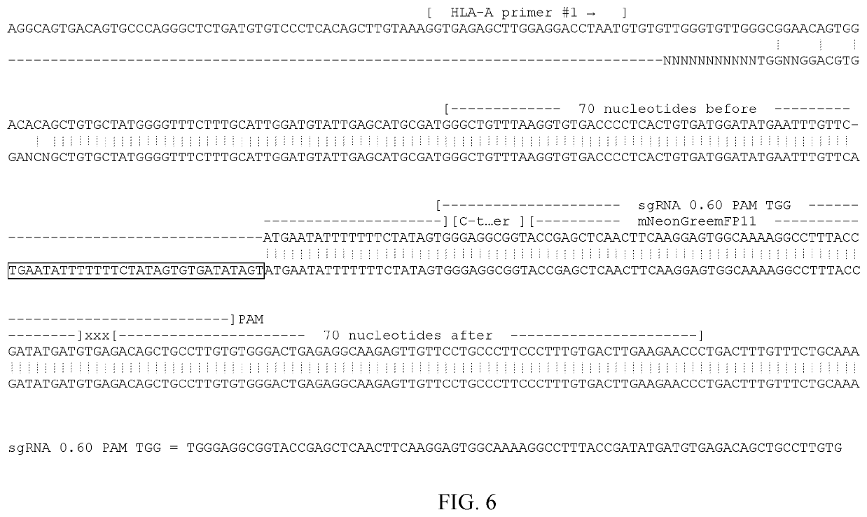

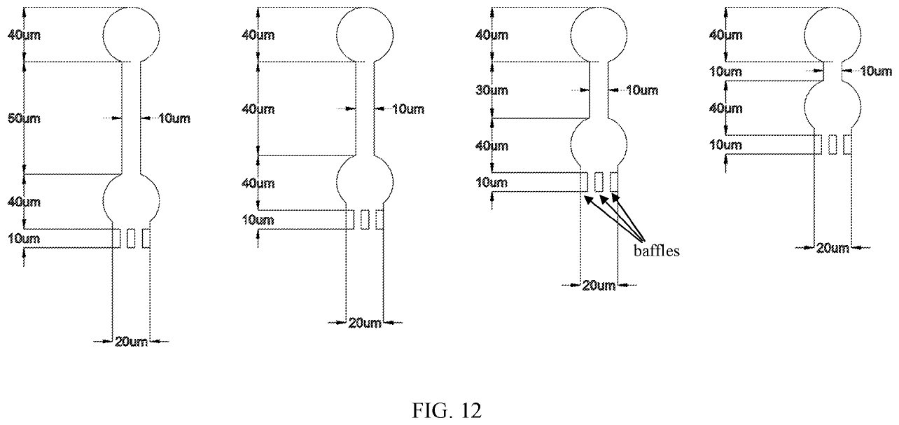

A transwell system comprises a first microwell and a second microwell (one for an exosome donor cell and one for a recipient cell) connected by a microchannel (also termed exosome transport channel) (FIGS. 6 and 12). Each microwell is sized and adapted to hold a single mammalian cell and sufficient media to culture the cell.

In some embodiments, each microwell is about 0.125 nL in volume, equivalent to a typical density (1×10⁶ cells/mL) for cell culture

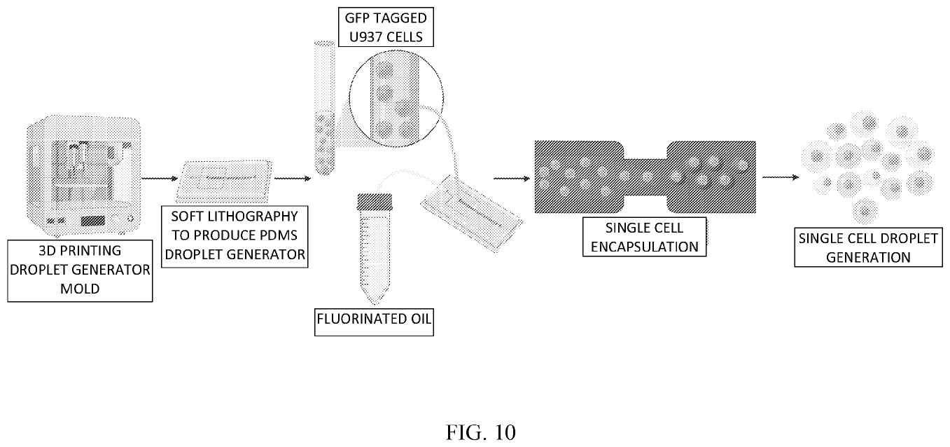

Each microwell forms an essentially cylindrical shape that is about 10 μm to about 500 μm in diameter and about 10 μm to about 500 μm in depth. The base (i.e., floor or bottom) of each microwell is essentially flat. In some embodiments, the base of each microwell comprises a microscope coverslip. The top of the microwell can be open to permit placement of a mammalian cell into the microwell, such as by single-cell droplet (e.g., FIG. 10). The microchannel is sized and adapted such that it is in fluid connection with and connects the first microwell and the second microwell. Fluid, such as growth media, but not the cells in the microwells, can flow or diffuse from one microwell to the other. Exosomes from a cell in one microwell (the exosome donor cell) can travel (such as by diffusion) to a cell in the other microwell (recipient cell) via the microchannel. The transwell system allows for secretome exchange and transport for communications between paired cells in the microwell via the microchannel. Such secretome exchange and transport could be two directional or one directional via passive mass diffusion due to culture medium concentration gradient.

In some embodiments, each microwell is about 10 μm, about 20 μm, about 30 μm, about 40 μm, about 50 μm, about 60 μm, about 70 μm, about 80 μm, about 90 μm, about 100 μm, about 200 μm, about 300 μm, about 400 μm, or about 500 μm in diameter. In some embodiments, each microwell is 40+10 μm or 40+5 μm in diameter. In some embodiments, each microwell is about 40 μm diameters in diameter. In some embodiments, each microwell is about 10 μm, about 20 μm, about 30 μm, about 40 μm, about 50 μm, about 60 μm, about 70 μm, about 80 μm, about 90 μm, about 100 μm, about 110 μm, about 120 μm, about 150 μm, about 200 μm, about 300 μm, about 400 μm, or about 500 μm in depth. In some embodiments, each microwell is 100±10 μm in depth. In some embodiments, each microwell is about 100 μm diameters in depth.

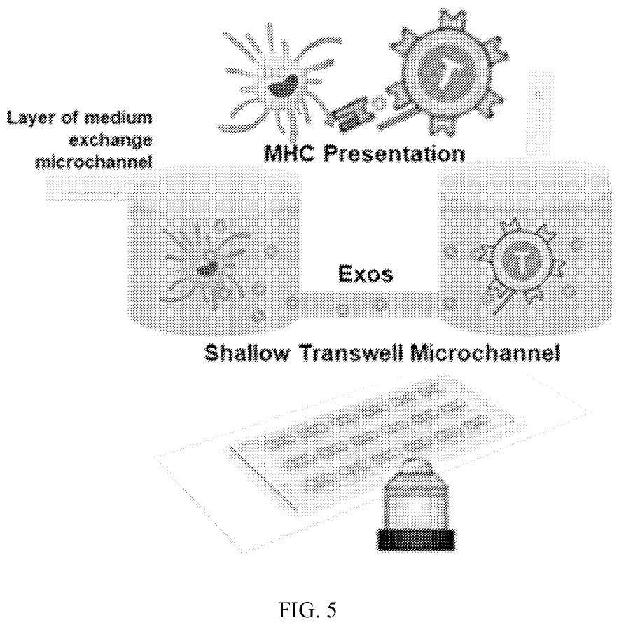

The microchannel is about 5 μm to about 15 μm in width and about 5 μm to about 15 μm in depth. In some embodiments, the microchannel is about 10 μm in width and about 10 μm in depth. The microchannel can be about 10 to about 60 μm in length. In some embodiments, the microchannel is about 10 μm, about 20 μm, about 30 μm, about 40 μm, about 50 μm, or about 60 μm in length (FIG. 12). In some embodiments, the microchannel is essentially linear, connecting the first microwell and the second microwell via a straight line. The microchannel is connected at the base of the microwells, such that the bottoms of the microwells and the bottom of the microchannel essentially lie in a plane (FIG. 5).

In some embodiments, a transwell system further comprises one or more perfusion channels in fluid connection with one or both microwells of the transwell system. The one or more perfusion channels can be connected to a single microwell in the transwell system or to both microwells in the transwell system. The perfusion channel is adapted to allow media, nutrients or other agents to be delivered the cells in the transwell system. In some embodiments, the perfusion channel is adapted to allow media, nutrients or other agents to be delivered the cells in the transwell system while maintaining a nutrient gradient between the two microwells in the transwell system. In some embodiments, hydrodynamic flow of fluid through the perfusion channel can be used to control a gradient of nutrients or other agents within the transwell system.

A perfusion channel can be about 10 μm to about 40 μm in width and about 10 μm to about 500 μm in depth. In some embodiments, the depth of the perfusion channel is the same as the depth of the microwell to which it is connected. In some embodiments, a perfusion channel is about 10 μm, about 15 μm, about 20 μm, about 25 μm, about 30 μm, about 35 μm, or about 40 μm in width. In some embodiments, a perfusion channel is about 10 μm, about 15 μm, about 20 μm, about 30 μm, about 40 μm, about 50 μm, about 60 μm, about 70 μm, about 80 μm, about 90 μm, about 100 μm, about 200 μm, about 300 μm, about 400 μm, or about 500 μm in depth. In some embodiments, a perfusion channel is about 20 μm in width (FIG. 12). In some embodiments, a perfusion channel is about 100 μm in depth. If more than one perfusion channel is present, the different perfusion channels can have different widths and/or depths. In some embodiments, the perfusion channels are connected to the top of the microwells, such that the top of the microwell and the top of the microchannel essentially lie in a plane. The perfusion channels can have one or more baffles adapted to prevent cells from being washed out of the transwells (FIG. 12). The baffles can be about 4 μm to about 10 μm in width, about 4 to about 20 μm or more in length, and the same height as the depth of the perfusion channel. The distance between the baffles can be about 4 μm to about 10 μm. In some embodiments, the baffles are about 4 μm in width and about 10 μm in length. The distance between the baffles is about 4 μm. The perfusion channels can be in fluid connection with a media inlet. In some embodiments, the media inlet is about 1500 μm diameter.

The transwell system is configured to permit fluorescence microscopic analysis of the cells. In some embodiments, the transwell system comprises a microscope coverslip (about 0.15 mm thick) base. The coverslip base can form the bottom of the microwells and the bottom of the microchannel.

### IV. Transwell System Array

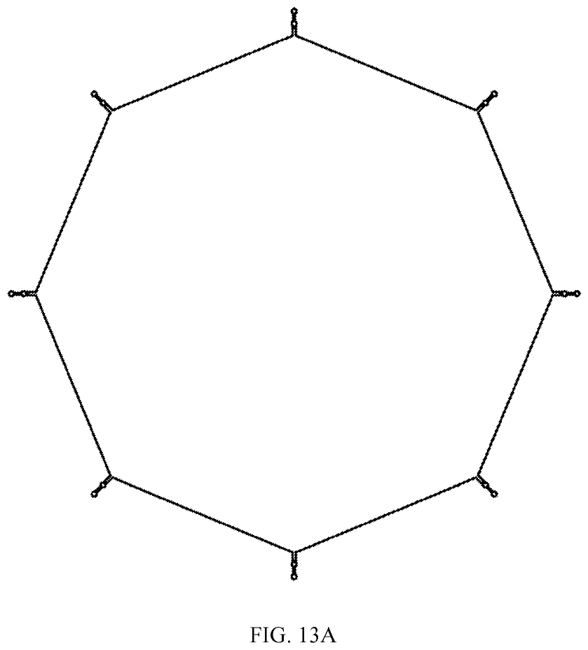

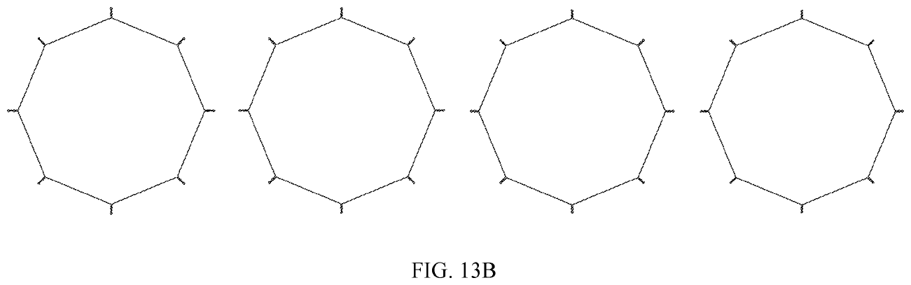

Two or more transwell systems can be combined in a transwell system array. A transwell system array comprises 2 or more transwell systems connected by one or more perfusion channels. A transwell system array can comprise 2, 3, 4, 5, 6, 7, 8, 9, 10, or more transwell systems connected by one or more perfusion channels. In some embodiments, a transwell system array comprises 2-24 transwell systems connected by one or more perfusion channels. The 2 or more transwell systems in a transwell system microarray can be connect by the one or more perfusion channels in series or in parallel. The 2 or more transwell systems in a transwell system microarray can be connected in any pattern. In some embodiments, the 2 or more transwell systems in a transwell system microarray can be connected in a linear array. In some embodiments, 3 or more transwell systems in a transwell system microarray can be connected a generally circular array (including, e.g., and triangle, square, pentagon, hexagon, heptagon, octagon (FIG. 13A), etc.). The one or more perfusion channels can connect to a single microwell in each transwell system or both microwells in each transwell system. In some embodiments, the single perfusion channel connects to single microwell in each transwell system in the transwell system array.

Each transwell system in a transwell system array can have a microchannel having the same length. Alternatively, the transwell systems in a transwell system array can have microchannels having different lengths.

In some embodiments, a transwell system array comprises 2-24 transwell systems connected to a perfusion channel. In some embodiments, a transwell system array comprises 8 transwell systems connected to a perfusion channel. In some embodiments, the 8 transwell systems are arranged in an octagon and the perfusion channel is in fluid connection with a single transwell in each transwell system (FIG. 13A).

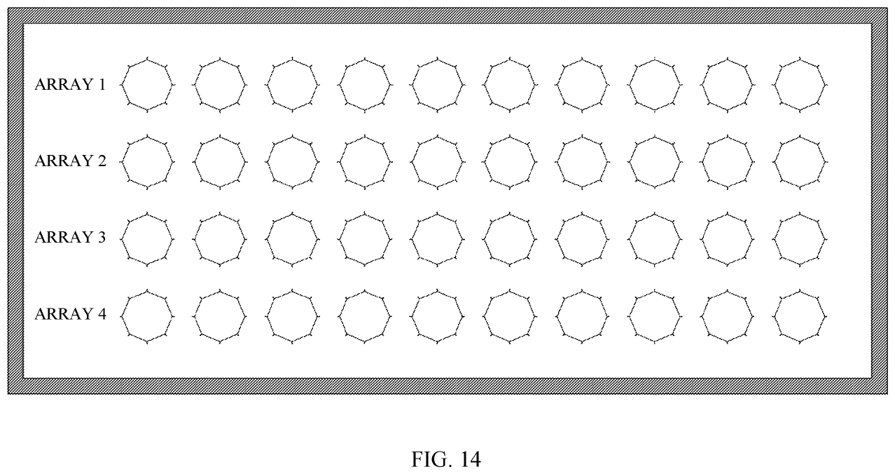

Multiple transwell systems arrays can be combined on a single unit, chip, or wafer (FIG. 14). In some embodiments, two or more rows each having two or more transwell system arrays are fabricated on a single unit. In some embodiments, two or more rows each having two or more transwell system arrays having microchannels of varying lengths are fabricated on a single unit. In some embodiments, each transwell system within each transwell system array can have microchannels of varying lengths. In some embodiments, each transwell system array within each row of transwell system arrays can have microchannels of varying lengths. In some embodiments, the transwell system arrays of each row of transwell system arrays can have microchannels of varying lengths. In some embodiments, four rows of 10 transwell system arrays having microchannels of varying lengths are fabricated on a single unit.

The transwell systems and transwell system arrays can be fabricated using any suitable material. In some embodiments, the transwell systems and transwell system arrays can be fabricated on silicon wafers via reactive ion etching and soft lithography. Soft lithograph can be, but is not limited to PDMS soft lithography.

In some embodiments, a PDMS chip with open culture chambers (first and second microwells) is covered by a second layer of PDMS containing microchannels interconnecting the culture chambers. In some embodiments, the bottom face of a molded PDMS transwell system is bonded to a microscope coverslip (0.15 mm thick) to form the closed micro fluid channel for high-resolution inverted microscopic imaging. The transwell system (or transwell system array) is configured to be compatible with visualization using a fluorescence microscope and high-throughput live-cell imaging.

The described transwell systems and transwell arrays can be provided as microscopic stage top cell culture and imaging cartridge devices for super resolution single cell imaging.

### V. Donor cell

Exosomes are produced by all or nearly all mammalian cells Therefore, nearly any mammalian cell can be used as a donor cell. The donor cell can be, but is not limited to, a dendritic cell, a macrophage, a blood fibrocyte, a cancer cell (e.g., Leukemia), or a mesenchymal stem cell.

The donor cell can be modified to express an exosome tag (Mittelbrunn et al. “Unidirectional transfer of microRNA-loaded exosomes from T cells to antigen-presenting cells.” Nat Commun. 2011 April; 2:282). In some embodiments, the exosome tag is detectable using fluorescence microscopy. The tag can be a detectable peptide or protein. The detectable peptide or protein can be, but is not limited to, a SunTag or a fluorescent protein. The fluorescent protein can be, but is not limited to, Green fluorescent protein (GFP), GFP-like proteins, modified GFPs, GFP derivatives, eGFP, eqFP611, Dronpa, TagRFPs, KFP, EosFP/IrisFP, Dendra, m Venus, mCherry, emerald GFP, superfolder GFP, Azami Green GFP, TagGFP, Turbo GFP, AcGFP, ZsGreen GFP, T-sapphire GFP, blue fluorescent protein, FBFP, FGFP2, Azurite BFP, mTagBFP, Cyan fluorescent protein (CFP), SCFP, mECFP, Cerulean CFP, mTurquoise CFP, CyPET CFP, AmCyan1 CFP, Modori-Ishi Cyan CFP, TabCFP, mTFP (Teal), yellow fluorescent protein (YFP), Topax YFP, Venus YFP, mCitrine YFP, YPet YFP, TagYFP, PhiYFP, Zs Yellow YFP, mBanana YFP, orange fluorescent protein (OFP), Kusabira Orange OFP, Kusabira Orange2 OFP, mOrange OFP, mOragne2 OFP, dTomato OFP, dTomato-Tandem OFP, TagRFP OFP, TagRFP-T OFP, DsRed OFP, DsRed2 OFP, DsRed-Express (T1) OFP, DsRed-Monomer OFP, m Tangerine OFP, Red fluorescent protein (RFP), mRuby RFP, mApple RFP, mStrawberry RFP, AsRed2 RFP, mRFP1 RFP, JRed RFP, mCherry RFP, HcRed1 RFP, mRaspberry RFP, dKeima-Tandem RFP, HcRed-Tandem RFP, mP1um RFP, AQ143 RFP, GFP11 (FP11), and sfCherry11. In some embodiments, tandem fluorophore labeling is used. Tandem labeling can be used to increase brightness of exosomes to facilitate single cell single vesicle imaging. A tandem label can include two or more copies of an eleventh β strand of a GFP or sfCherry fluorescent protein, (GFP11, and sfCherry11, respectively; Kamiyama D et al. “Versatile protein tagging in cells with split fluorescent protein.” Nat Commun. 2016;7:11046.). In some embodiments, up to 700-nt DNA coding sequences at specific genomic loci can be used.

The donor cell can be modified to express an exosome tag by introducing a nucleic acid sequence encoding the exosome tag into the genome of the donor cell. In some embodiments, an endogenous gene in the donor cell that is known to be incorporated into exosomes is tagged. A tagged protein comprises a fusion between the endogenous protein and the tag. The tagged protein can be formed by introducing the nucleic acid encoding the tag into the genomic of the donor cell at the location of the sequence encoding the protein to be tagged such that the endogenous gene is engineered (modified) to encode and express the tagged protein.

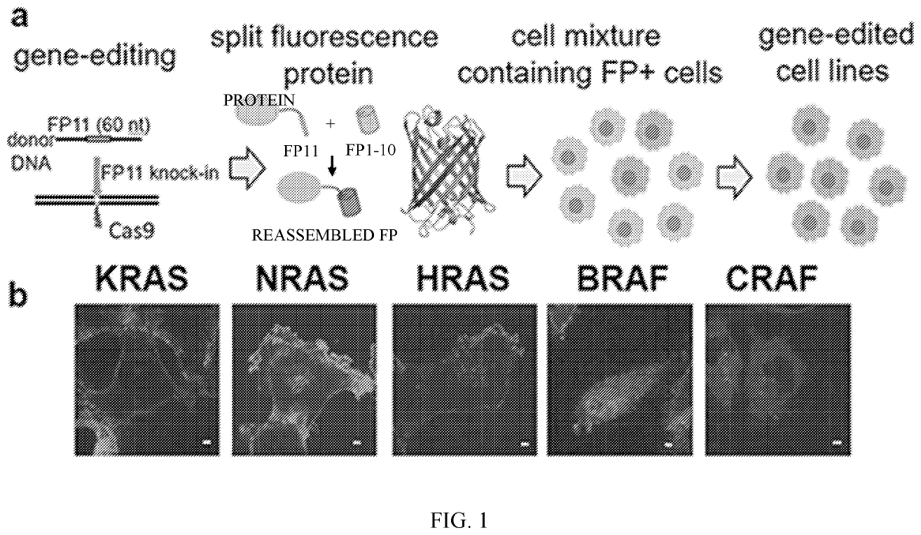

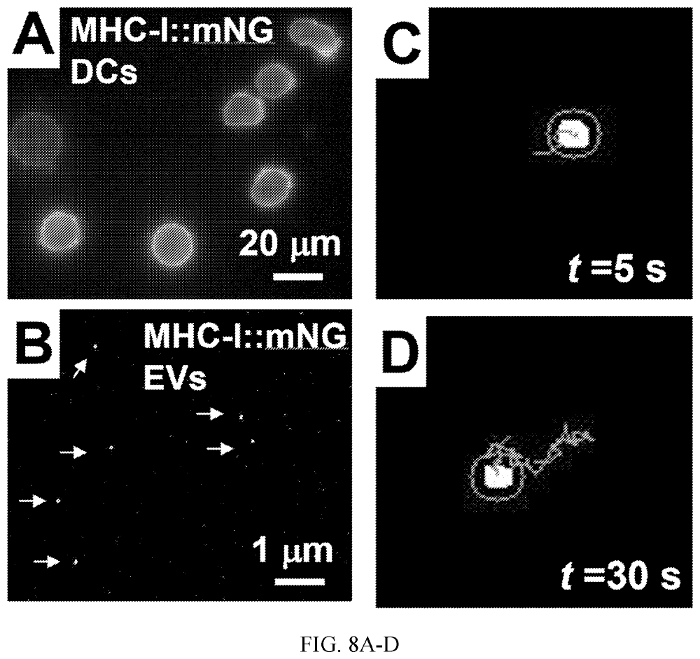

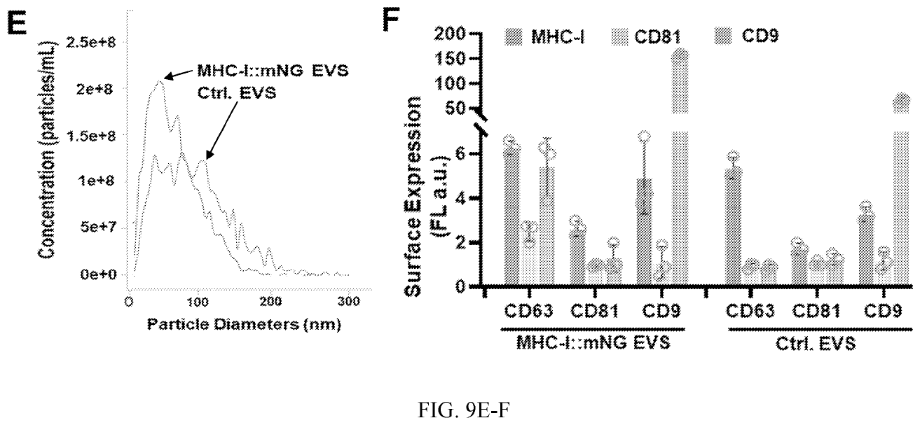

In some embodiments, the endogenous protein to be tagged is a membrane-bound protein. The endogenous protein to be tagged may be specific to the donor cell. Endogenous proteins that may be tagged include, but are not limited to, MHC (class I and II), ESCRT, tetraspanins, heat shock proteins, adhesion molecule, cytoskeletal proteins, TIM4, CD9, HSPA8, PDCD6IP, GAPDH, ACTB, ANXA2, CD63, SDCBP, ENO1, HSP90AA1, TSG101, PKM, LDHA, EEF1A1, YWHAZ, PGK1, EEF2, ALDOA, HSP90AB1, ANXA5, FASN, YWHAE, CLTC, CD81, ALB, VCP, TPI1, PPIA, MSN, CFL1, PRDX1, PFN1, RAP1B, ITGB1, HSPA5, SLC3A2, HIST1H4A, GNB2, ATP1A1, YWHAQ, FLOT1, FLNA, CLIC1, CDC42, CCT2, A2M, YWHAG, TUBA1B, RAC1, LGALS3BP, HSPA1A, GNAI2, ANXA1, RHOA, MFGE8, PRDX2, GDI2, EHD4, ACTN4, YWHAB, RAB7A, LDHB, GNAS, TFRC, RAB5C, ARF1, ANXA6, ANXA11, ACTG1, KPNB1, EZR, ANXA4, ACLY, TUBA1C, RAB14, HIST2H4A, GNB1, UBA1, THBS1, RAN, RABSA, PTGFRN, CCT5, CCT3, BSG, AHCY, RAB5B, RAB1A, LAMP2, ITGA6, HIST1H4B, GSN, FN1, YWHAH, TUBA1A, TKT, TCP1, STOM, SLC16A1, and RAB8A. In some embodiments, the tagged endogenous protein comprises: a MHC class I protein, a MHC claim II protein, ESCRT, a tetraspanin, a heat shock protein, an adhesion molecule, a cytoskeletal protein, or TIM4. In some embodiments, the tagged endogenous protein comprises a MHC class 1 protein (FIGS. 1-4, 7-9). The MHC class 1 protein can be, but is not limited to HLA-A, HLA-B, or HLA-C.

Although the fluorescence protein fusions such as plasmid-based GFP fusion protein can mark the proteins for tracking, plasmid systems often aggressively overexpress proteins, causing deviations in the natural cellular physiological states. Labeling native proteins at the genome level avoids such deviations. In addition, cell-to-cell gene expression in the native genomic context has a narrower and specific distribution compared to transgene or transfection (plasmid)-based or protein expression system.

Methods for introducing a nucleic acid sequence into a genomic sequence are known in the art and include, but are not limited to, CRISPR editing (Wilson et al. “Labeling Endogenous Proteins Using CRISPR-mediated Insertion of Exon (CRISPIE)” Bio Protoc. 2022 12(5):e4343). In some embodiments, the nucleic acid sequence encoding the exosome tag is inserted into the genome of a donor cell using CRISPR. The use of CRISPR to form the tagged endogenous protein decreases artificial effects of overexpression of plasmid expressed tagged proteins on the exosome donor cell or the resultant exosomes (Strohmeier K et al. “CRISPR/Cas9 Genome Editing vs. Over-Expression for Fluorescent Extracellular Vesicle-Labeling: A Quantitative Analysis,” Int. J. Mol. Sci. 2022, 23(1):282; Ye Y et al. “In Vivo Visualized Tracking of Tumor-Derived Extracellular Vesicles Using CRISPR-Cas9 System,” Technology in Cancer Research & Treatment, 2022, 21:1-12; and de Jong OG et al. “CRISPR-Cas9-based reporter system for single-cell detection of extracellular vesicle-mediated functional transfer of RNA,” Nature Communications 2020; 11:1113).

Modification of the protein to be tagged and the site of the genome locus minimizes the interference on natural cellular biogenesis of exosomes including selective molecular sorting.

In some embodiments, the endogenous protein is tagged such that the tag is on the interior of exosomes produced by the donor cell. By positioning the tag such that the tag in present in the exosome interior, obstruction of the exosome in minimized and exosome surface immunogenicity is maintained, leading to retention of native exosome function

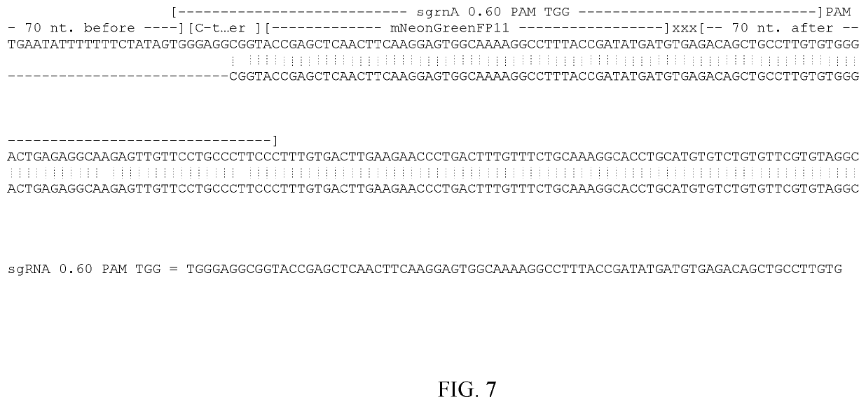

In some embodiments, the donor cell is tagged by tagging MHC-I at the C-terminus such that the fluorescent tag is attached to the MHC-I cytoplasmic tail and thereby present in the exosome interior compartment (FIG. 6-7).

### VI. Recipient (Target) Cell

The target or recipient cell can be any mammalian cell. The recipient cell can be, but is not limited to, a T cell, a keratinocyte, a tumor infiltrating T cell, or a tumor cell, or a natural killer cell. In some embodiments, the recipient cell is a T cell.

### VII. Methods of Use

The described transwell systems and methods provide means to image natural cellular biogenesis and trafficking of exosomes. The described transwell systems and methods further provide solutions for studying exosome molecular targets in high precision and high throughput.

To analyze exosome-mediated communication between a first cell and a second cell, the first cell is placed in and cultured in one well of a transwell system and a second cell is placed in and cultured in the other well of a transwell system. The first cell comprises a tagged endogenous protein, wherein the endogenous protein is incorporated into exosomes produced by the first cell. Biogenesis of exosomes, shedding of exosomes, and diffusion of exosomes to the second cell are analyzed by fluorescence microscopy. Changes in gene expression in the second cell following targeting of exosomes to the second cell can be analyzed via single cell transcriptome analyses.

In some embodiments, the described transwell systems and methods can be used to analyze and track exosome precursor packaging, exosome shedding, exosome transport and exosome delivery to target cells in one integrated workflow.

In some embodiments, the described transwell systems and methods can be used to analyze non-contact, long-distance cellular communication (including immune activation) between a single donor-recipient cell pair (e.g., dendritic cell to T cell).

In some embodiments, the described transwell systems and methods can be used to image exosome tracking dynamics.

In some embodiments, the described transwell systems and methods can be used to analyze single donor-recipient cell pair (e.g., dendritic cell to T cell) communication via exosomes with statistical significance.

In some embodiments, the described transwell systems and methods can be used to analyze the molecular mechanism controlling exosome makeup.

In some embodiments, the described transwell systems and methods can be used to analyze intercellular immune communication.

In some embodiments, the described transwell systems and methods can be used in the development of precision cancer immunotherapy.

In some embodiments, the described transwell systems and methods can be used to analyze paired single cell events such as tumor cell-T cell immune checkpoint inhibition, or tumor cell-natural killer cell interactions. The tumor cell and/or T cell can be, but is not limited to, a patient-derived tumor cell and/or T cell.

In some embodiments, the described transwell systems and methods can be used to analyze bi-directional tracking of exosomes between paired single cells, or triangular tracking between single macrophage, tumor cell, and T cell.

In some embodiments, the described transwell systems and methods can be used to screen or fish for T cell with heterogeneous TCR repertoires for neoantigen discovery via exosome MHC antigen presentation pathway. The T cell can be, but is not limited to, a patient-derived T cell.

In some embodiments, the described transwell systems and methods can be used to analyze changes in gene expression in a recipient cell in response to exosomes.

The sensitivity and precision offered by described transwell systems and methods are superior to current Elit Spot platform on single cell immune analysis.

Although the invention has been described in detail for purposes of clarity of understanding, certain modifications may be practiced within the scope of the appended claims. All publications, accession numbers, web sites, patent documents and the like cited in this application are hereby incorporated by reference in their entirety for all purposes to the same extent as if each were so individually denoted. To the extent different information is associated with a citation at different times, the information present as of the effective filing date of this application is meant. Unless otherwise apparent from the context any element, embodiment, step, feature or aspect of the invention can be performed in combination with any other.

### EXAMPLES

### Example 1. CRISPR Cas9 Endogenous Protein Tagging of Endogenous MHC-I for Tracking Natural Cellular Packaging of Exosomes at the Single Cell Single Vesicle Level

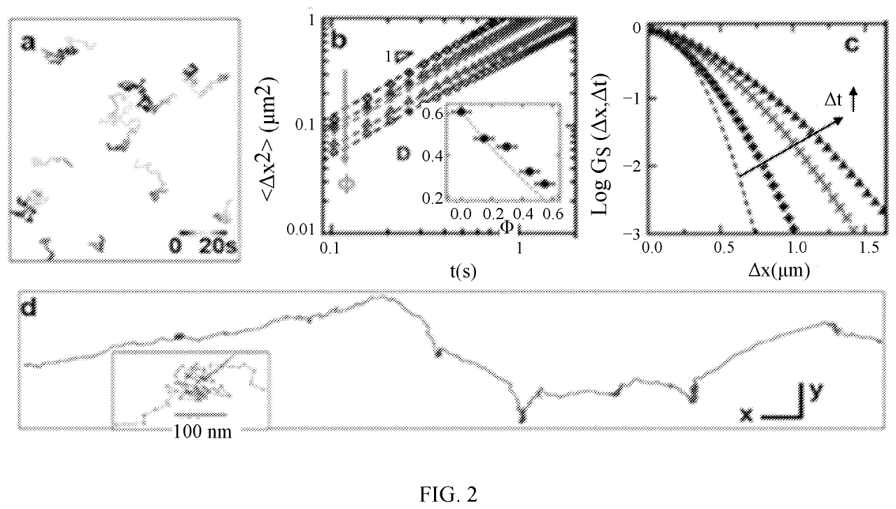

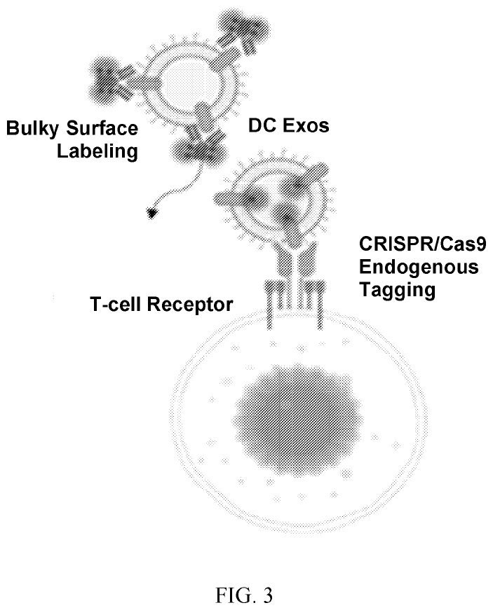

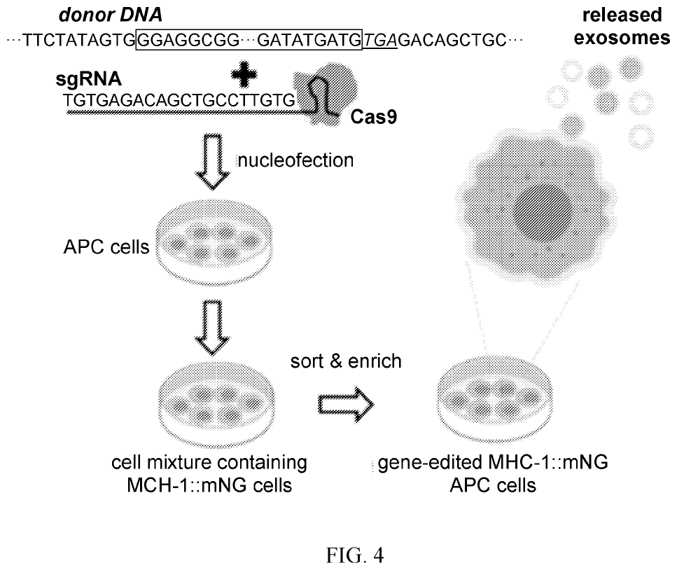

A. CRISPR/Cas9 endogenous tagging has been used to tag numerous proteins, including KRAS, NRAS, HRAS, BRAF, and CRAF, in various cell types, as shown in FIG. 1.
B. A single particle tracking algorithm and a wavelet-based universal thresholding algorithm for automatically distinguishing between “moving” and “stationary” phases of trajectories has previous been shown (as illustrated in FIG. 2). Those algorithms will be directly applicable to the proposed work for tracking exosome release, transport, docking to recipient T cells.
C. Single cell imaging of exosome biogenesis and secretion dynamic is challenging, due to limited microscopic labeling and sensing ability. Conventional antibody tagging or dye labeling introduces bulky obstruction or surface alteration that can block receptor interactions at the exosome surfaces. This is especially problematic in functional studies of exosome-induced immune cell activation where the exosome surface protein MHC-I needs to form a tight binding interface with T-cell receptor (TCR) (as illustrating in FIG. 3). To overcome this challenge and retain the natural biogenesis and immunogenicity of exosomes, the described CRISPR/Cas9 endogenous protein “knock-in” tagging scheme provides for natural exosome formation in the parent cell, interaction with the target cell, and real time fluorescence tracking of secretion dynamic, transport and presentation to recipient cells. Our imaging spatial resolution is 30 nm which is sufficient for tracking single exosomes with typical size of ˜150 nm.
D. We have demonstrated efficient and scarless genomic insertion of fluorescence tag coding sequence at various genes of interest in diverse cell lines as shown in FIG. 1.
E. Overall experimental design to tag MHC-I exosomes is a two-step strategy: Human dendritic cell line differentiated from THP-1 are MHC-I fluorescently tagged in the genome using CRISPR. MHC-I is tagged at the C-terminus to such that the fluorescent tag is attached to the MHC-I cytoplasmic tail and thereby present in the exosome interior compartment (as illustrated in FIG. 3). Fluorescent-MHC-I packed exosomes are released from the knock-in parent cells for high resolution microscopic analysis. By positioning the tag such that the tag in present in the exosome interior, obstruction of the exosome in minimized, leading to retention of native exosome function, such as immunogenicity. The detailed schematic workflow from generating tagged DCs to probing MHC-I containing exosomes is shown in FIG. 4 including the design of the fluorescence tag and sgRNA sequence in MHC-I isoform HLA-A. The other two MHC-I isoforms HLA-B and HLA-C can be similarly designed to stably express fluorescence tagged MHC-I proteins in DC cell line for exosome packaging. Other proteins can be similarly tagged. The selection of protein to be tagged can depend of the donor cell and exosome function. The non-tagged DC cell line and secreted exosomes will serve as the control group to evaluate the immunogenicity from tagged DCs and their exosomes. The functional assay of tagged DCs and exosomes will be assessed in terms of immune marker expression, secretion dynamics, size, and presentation to recipient T cells (e.g., Primary CD8+ Cytotoxic T Cells from ATCC).

Proof of concept is done using an mNeonGreen-MHC protein

### Example 2. Double Knock-In Donor Cells

To show that the dynamics of MHC-I specific exosome biogenesis mimics the in vivo process under the natural cellular regulatory control, we will implement CRISPR-based double “knock-in” strategy to achieve two-color labeling using a second “knock-in” step to endogenously tag CD63 (a well-documented exosome membrane marker) with mCherry in the MHC-I::mNeonGreen DC cell line. The double knock-in provides for simultaneous imaging of both MHC-I and specific endomembrane compartments to dissect the MHC-I exosome biogenesis pathway, and ensure the specific tracking of exosomes. Live cells are imaged in real time to record co-localization and intracellular transport of MHC-I endomembrane vesicles and their dynamics for presentation. We have written custom algorithms (Asmin Tulpule JG et al. “Cytoplasmic protein granules organize kinase-mediated RAS signaling.” bioRxiv. 2019.; Guan J et al. “Even hard-sphere colloidal suspensions display Fickian yet non-Gaussian diffusion.” ACS Nano. 2014;8(4):3331-6; Chen K et al. “Diagnosing heterogeneous dynamics in single-molecule/particle trajectories with multiscale wavelets.” ACS Nano. 2013;7(10):8634-44; and “Guan J et al. Modular stitching to image single-molecule DNA transport.” J Am Chem Soc. 2013;135(16):6006-9) to conduct quantitative image analysis specifically relevant to the images acquired (as illustrated in FIG. 2), including identification of subcellular co-localization with adaptive thresholding and dynamics in intracellular transport captured at high spatiotemporal resolution. These image analysis algorithms are directly transferrable to achieve exosome biogenesis tracking at the single cell level.

A stable dendritic cell line that constitutively expresses a MHC-I-mNeonGreen2 protein via modification at the MHC-I genomic locus is generated. After harvesting exosomes released from this tagged cell line, the efficiency of MHC-I packaging into exosomes and their immunogenicity is assessed compared with control non-modified DCs and their secreted exosomes using immunogenicity assays as described in Hong SR et al. (“Immunogenic potency of engineered exosomes for prevention of respiratory syncytial virus.” The Journal of Immunology. 2020;204:245.22).

A stable dendritic cell line that constitutively expresses a MHC-I-NeonGreen2 at MHC-I protein via a modification at the MHC-I genomic locus and an CD63-mCherry protein via a modification at the CD63 genomic locus is generated.

Live-cell imaging with high spatiotemporal resolution and quantitative imaging will be used to monitor the biogenesis of exosomes carrying MHC-I, analyze MHC-I sorting and intracellular transport at various exosome biogenesis stages. Labeling of CD63 in combination with MHC-I will enable analysis of MHC-I concentration and progression of exosome formation in the multivesicular bodies.

The ability of MHC-I tagged exosomes and native (untagged exosomes) to activate and modulate immune function of recipient T cells will be analyze by cellular immune profiling.

In some embodiments, MHC-1 will be labeled using tandem labeling to increase the brightness of exosomes and facilitate single cell single vesicle imaging.

While the example above utilizes mNeonGreen-MHC protein as the tagged protein, other tags and endogenous proteins can readily be made using similar methods.

### Example 3. Microfluidic High-Throughput Single Cell Transwell Array Which Enables Real-Time Tracking of Exosome Immunity Regulation in Paired Donor-Recipient Immune Cells

Studying paired single donor-recipient immune cells can be used to identify statistically effective key molecular modulators carried by exosomes. Such study is not possible with previously described systems. The described microfluidic-based single-cell transwell system for culturing single donor (e.g., dendritic) cell and target (e.g., T) cell pairs and real time tracking exosome mediated immune regulation in anti-tumor T cell activation, which can mimic in vivo long-range tumor immunity regulation (see FIG. 5). It has been reported that exosomes are essential in bidirectional cellular communication for regulating tumor microenvironment and associated tumor immunity (Wang T et al. “Functions of Exosomes in the Triangular Relationship between the Tumor, Inflammation, and Immunity in the Tumor Microenvironment.” J Immunol Res. 2019;2019:4197829. Epub 2019 Aug. 31; Valcz G et al. “Perspective: bidirectional exosomal transport between cancer stem cells and their fibroblast-rich microenvironment during metastasis formation.” NPJ Breast Cancer. 2018;4:18. Epub 2018 Jul. 25; Maia J et al. “Exosome-Based Cell-Cell Communication in the Tumor Microenvironment.” Front Cell Dev Biol. 2018;6:18. Epub 2018 Mar. 9; Dioufa N et al. “Bi-directional exosome-driven intercommunication between the hepatic niche and cancer cells.” Mol Cancer. 2017;16(1):172. Epub 2017 Nov. 16).

A microfluidic interconnected transwell culture array is fabricated using 3D PDMS molding. An array of single-cell transwells will be designed and fabricated to host 24 single DC-T cell pairs. Each transwell will host a single CRISPR endogenous MHC-I tagged DC and a paired single T cell separated by a shallow microchannel. The depth of the channel between the single-cell wells is ≤5 μm, which only allows the secretome exchange and transport for bidirectional communications between paired cells. Because the MHC-I peptide antigenic presentation relies on the membrane receptor contact with T cells either by dendritic cells or dendritic cell derived exosomes, other secretome factors that present and exchange through the microchannels will not interfere with exosome MHC presentation. The PDMS chip with open culture chambers is covered by a second layer of PDMS containing microchannels interconnecting culture chambers for medium exchange and precision control of gradient via hydrodynamic flow. The bottom face of molded PDMS transwell is bonded to a microscope cover slip (0.15 mm thick) to form the closed microchannel for high-resolution inverted microscopic imaging. Thus, the footprint and setup are compatible with fluorescence microscope for high-throughput super resolution live cell imaging. The full dynamic process of exosome biogenesis, release, transport, and internalization can be profiled and analyzed in one workflow.

The described transwell systems will provide a high-spatiotemporal resolution, live cell imaging microfluidic platform, which enables the single cell imaging and tracking single exosome dynamics at the scale. This platform is generic for studying other single-cell level events using imaging approach such as tumor cell-T cell for immune checkpoint inhibition.

The described transwell systems and methods can be used to study exosome mediated long range communication between cells.

The described transwell systems can be used to study the behaviors and mechanisms that regulate exosome-mediated long-range immune activation in tumor microenvironments, such as pancreatic tumor microenvironments.

The described transwell systems can be used to study the correlation of exosome-based molecular modulators with cellular-level immunity activation.

The described transwell systems can be used to study the molecular mechanisms in exosome packaging, which in turn could develop molecularly tailored exosomes for reprogramming intercellular immune communication as the novel exosome therapy.

### Example 4

A double knock-in dendritic cell line is generated that contains both MHC-I::mNeonGreen and CD63::mCherry. The tagged dendritic cells are then placed in the paired-cell microfluidic device and efficiency of packaging of MHC-I::mNeonGreen and CD63::mCherry is verified. Single vesicle tracking of MCH-I exosomes from the dendritic cells is also verified. Intracellular and intercellular transport, T cell internalization, and immunogenicity and functionality of exosomes is then characterized.

### Example 5

CRISPR is used to insert a fluorescence protein tag at a genomic locus (e.g., MHC-I (HLA-A) of exosome donor cells (e.g., THP-1 differentiated dendritic cells). The tag can be inserted, for example, at the C terminus of HLA-A, right before the stop codon. Using fluorescence sorting, selecting for successful gene-editing events and correct open reading frame insertion, it is anticipated that off target CRISPR effects will be minimized. It is anticipated that >10 Reads Per Kilobase of transcript, per Million mapped reads is required. Since MHC-I is highly expressed in DCs in general, thus, sufficient tagging sensitivity enables the single vesicle tracking. In some embodiments, tandem-fluorophore labeling is used to further enhance the imaging sensitivity. Because the tags are integrated at the genomic level, the tagged cell lines will stably express the tagged protein. The expected 30 ms temporal resolution and 30 nm or better spatial resolution allows single vesicle tracking at the single cell level, and co-localization between MHC-I and CD63 in multivesicular bodies with ≥95% accuracy.

### Example 6. Transwell System Array

A transwell system array was fabricated to host 8 single Dendritic cell/T cell pairs. Each microwell in the transwell system array was about 40 μm in diameter. A single CRISPR endogenous MHC-I tagged DC was placed in the first microwell and a single T cell was placed in the second microwell of each transwell system. The transwell systems had microchannels of varying lengths.

U937 knock-in tagged dendritic cells are paired with primary CD8+ cytotoxic T cells. The PDMS chip with open culture chambers will be covered by the second layer of PDMS containing microchannels interconnecting culture chambers for medium exchange and precision control of gradient via hydrodynamic flow. The bottom face of molded PDMS transwell will be bonded to a microscope coverslip (0.15 mm thick) to form the closed microchannel for high-resolution inverted microscopic imaging. Thus, the footprint and setup are compatible with a fluorescence microscope for high-throughput live-cell imaging. A single-cell droplet can be used to dispense single cells into the transwell system (FIG. 5). The imaging and tracking of CRISPR tagged exosomes can be achieved in real-time by viewing the transwell chamber via high-spatiotemporal resolution microscope. Thus, the whole dynamic process of exosome biogenesis, release, transport, and internalization can be profiled and analyzed in one workflow.

### Example 7. Live Cell Imaging

U937 dendritic cells tagged with FP-11 MHC-I were prepared as described and above individual cells were placed into a well of a described transwell system. Time-lapse, live-cell fluorescence microscopic imaging of the cells was then performed.

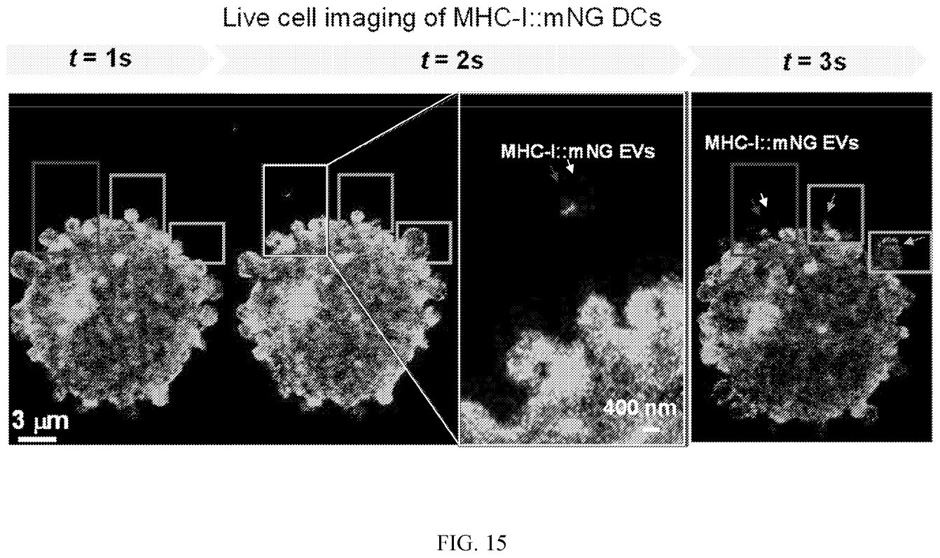

Time-lapse, live-cell fluorescence microscopic imaging of a U937 dendritic cell tagged with FP-11 MHC-I is shown in FIG. 15. Along the millisecond time-frame, we captured the real-time exosome secretion from the tagged U937 model parent cells. Real-time tracking of exosomes, from biogenesis secretion to transport and antigenic presentation, was highly specific to MHC-I expression. We observed highly abundant MHC-I positive exosomes to transport to extracellular space in fast migration speed as show in FIG. 1.

It will be understood that the present invention has been described above by way of example only. The examples are not intended to limit the scope of the invention. Various modifications and embodiments can be made without departing from the scope and spirit of the invention.

1. A microfluidic transwell system for use in analyzing exosome interactions comprising:
(a) a first microwell sized and adapted to culture a single cell;
(b) a second microwell sized and adapted to culture a single cell; and
(c) a microchannel connecting and in fluid communication with the first microwell and the second microwell.

2. The microfluidic transwell system of claim 1, wherein the first microwell and the second microwell are about 10 μm to about 500 μm in diameter and about 10 μm to about 500 μm in depth.

3. The microfluidic transwell system of claim 1, wherein the first microwell and the second microwell are about 40 μm in diameter and about 100 μm in depth.

4. The microfluidic transwell system of claim 1, wherein the microchannel is about 5 μm to about 15 μm in width, and about 5 μm to about 15 μm in width, and about 10 to about 60 μm in length.

5. The microfluidic transwell system of claim 1, wherein the microchannel is about 10 μm in width and about 10 μm in width.

6. The microfluidic transwell system of any one of claims 1-5, wherein the microfluidic transwell system is configured to permit fluorescence microscopic analysis of a cell or exosome in the first microwell, the second microwell, and/or the microchannel.

7. The microfluidic transwell system of claim 6, wherein microfluidic transwell system further comprises a coverslip.

8. The microfluidic transwell system of claim 7, wherein the coverslip forms the bottom of the first microwell, the second microwell, and the microchannel.

9. The microfluidic transwell system of any one of claims 1-8, wherein the microfluidic transwell system further comprises one or more perfusion channels in fluid connection with the first microwell and/or the second microwell.

10. The microfluidic transwell system of claim 9, wherein the perfusion channel is about 10 μm to about 40 μm in width and about 10 μm to about 500 μm in depth.

11. The microfluidic transwell system of claim 10, wherein the perfusion channel is about 20 μm in width.

12. The microfluidic transwell system of any one of claims 9-11, wherein the perfusion channel is in fluid connection with a media inlet.

13. A microfluidic transwell system array comprising two of more microfluidic transwell systems as in any one of claims 1-8, wherein the two or more microfluidic transwell systems are connected by one or more perfusion channels.

14. The microfluidic transwell system array of claim 13, wherein the microfluidic transwell system array comprises 2-24 microfluidic transwell systems.

15. The microfluidic transwell system array of claim 14, wherein the one or more perfusion channels are 10 μm to about 40 μm in width and about 10 μm to about 500 μm in depth.

16. The microfluidic transwell system array of claim 14, wherein the perfusion channel is about 20 μm in width.

17. The microfluidic transwell system array of any one of claims 13-16, wherein the two or more microfluidic transwell systems contain microchannels having different lengths.

18. A microfluidic transwell system of any one of claims 1-17, wherein:
(a) the first microwell contains a single exosome donor cell expressing a tagged endogenous protein, wherein the tagged endogenous protein is incorporated into exosomes; and
(b) the second microwell contains a single recipient cell.

19. The microfluidic transwell system of claim 18, wherein the tagged endogenous protein comprises a CRISPR-generated genomic modification of the endogenous protein.

20. The microfluidic transwell system of claim 19, wherein the tag comprises a fluorescent protein or fragment thereof.

21. The microfluidic transwell system of claim 20, wherein the tag is configured to be present in the interior of the exosome.

22. A method of analyzing exosome-mediated cell-cell communication comprising:
a) placing an exosome donor cell into the first microwell of any one of claims 1-17, wherein the exosome donor cell expresses a tagged endogenous protein, wherein the tagged endogenous protein is incorporated into exosomes;
b) placing a recipient cell into the second microwell of any one of claims 1-17; and
c) analyzing the donor cell, the recipient cell, and/or an exosome produced by the donor cell vis fluorescence microscopy.

23. The method of claim 21, wherein the tagged endogenous protein comprises a CRISPR-generated genomic modification of the endogenous protein.

24. The method of claim 22, wherein the tag comprises a fluorescent protein.

25. The method of claim 23, wherein the tag is configured to be present in the interior of the exosome.

26. The method of any one of claims 22-25, wherein the donor cell comprises a dendritic cell.

27. The method of any one of claims 22-26, wherein the recipient cell comprises a T cell.

28. The method of any one of claims 22-25, wherein the donor cell or the recipient cell comprises a patient-derived cell.

29. The method of any one of claims 22-28, wherein analyzing exosome-mediated cell-cell communication comprises tracking exosome precursor packaging, exosome shedding, exosome transport and/or exosome delivery to target cell.

30. The method of any one of claims 22-28, wherein analyzing exosome-mediated cell-cell communication comprises analyzing non-contact, long-distance cellular communication.

31. The method of any one of claims 22-28, wherein analyzing exosome-mediated cell-cell communication comprises imaging exosome tracking dynamics.

32. The method of any one of claims 22-28, wherein analyzing exosome-mediated cell-cell communication comprises analyzing single donor-recipient cell pair communication via exosomes.

33. The method of any one of claims 22-28, wherein analyzing exosome-mediated cell-cell communication comprises analyzing intercellular immune communication.

34. The method of any one of claims 22-28, wherein analyzing exosome-mediated cell-cell communication comprises analyzing tumor cell-T cell immune checkpoint inhibition or tumor cell-natural killer cell interactions.

35. The method of any one of claims 22-28, wherein analyzing exosome-mediated cell-cell communication comprises analyzing changes in gene expression in a recipient cell in response to exosomes.

36. The method of any one of claims 22-28, wherein analyzing exosome-mediated cell-cell communication comprises screening for T cells with heterogeneous TCR repertoires for neoantigen discovery via exosome MHC antigen presentation pathway.

37. The method of any one of claims 22-28, wherein analyzing exosome-mediated cell-cell communication comprises single cell imaging and tracking of single exosomes.


SEQUENCE LISTING (8 sequences)

SEQ ID NO: 1 - DNA, 369 bp, synthetic construct
  source 1..369: mol_type = other DNA
aggcagtgacagtgcccagggctctgatgtgtccctcacagcttgtaaaggtgagagcttggaggacctaatgtgtgttgggtgttgggcggaacagtggacacagctgtgctatggggtttctttgcattggatgtattgagcatgcgatgggctgtttaaggtgtgacccctcactgtgatggatatgaatttgttcatgaatatttttttctatagtgggaggcggtaccgagctcaacttcaaggagtggcaaaaggcctttaccgatatgatgtgagacagctgccttgtgtgggactgagaggcaagagttgttcctgcccttccctttgtgacttgaagaaccctgactttgtttctgcaaa

SEQ ID NO: 2 - DNA, 323 bp, synthetic construct
  source 1..323: mol_type = other DNA
nnnnnnnnnnntggnnggacgtggancngctgtgctatggggtttctttgcattggatgtattgagcatgcgatgggctgtttaaggtgtgacccctcactgtgatggatatgaatttgttcatgaatatttttttctatagtgtgatatagtatgaatatttttttctatagtgggaggcggtaccgagctcaacttcaaggagtggcaaaaggcctttaccgatatgatgtgagacagctgccttgtgtgggactgagaggcaagagttgttcctgcccttccctttgtgacttgaagaaccctgactttgtttctgcaaa

SEQ ID NO: 3 - DNA, 77 bp, synthetic construct
  source 1..77: mol_type = other DNA
tgggaggcggtaccgagctcaacttcaaggagtggcaaaaggcctttaccgatatgatgtgagacagctgccttgtg

SEQ ID NO: 4 - DNA, 200 bp, synthetic construct
  source 1..200: mol_type = other DNA
tgaatatttttttctatagtgggaggcggtaccgagctcaacttcaaggagtggcaaaaggcctttaccgatatgatgtgagacagctgccttgtgtgggactgagaggcaagagttgttcctgcccttccctttgtgacttgaagaaccctgactttgtttctgcaaaggcacctgcatgtgtctgtgttcgtgtaggc

SEQ ID NO: 5 - DNA, 174 bp, synthetic construct
  source 1..174: mol_type = other DNA
cggtaccgagctcaacttcaaggagtggcaaaaggcctttaccgatatgatgtgagacagctgccttgtgtgggactgagaggcaagagttgttcctgcccttccctttgtgacttgaagaaccctgactttgtttctgcaaaggcacctgcatgtgtctgtgttcgtgtaggc

SEQ ID NO: 6 - DNA, 18 bp, synthetic construct
  source 1..18: mol_type = other DNA
ttctatagtgggaggcgg

SEQ ID NO: 7 - DNA, 21 bp, synthetic construct
  source 1..21: mol_type = other DNA
gatatgatgtgagacagctgc

SEQ ID NO: 8 - DNA, 20 bp, synthetic construct
  source 1..20: mol_type = other DNA
tgtgagacagctgccttgtg